(19) United States
(12) Patent Application Publication
(10) Pub. No.: US 2018/0221563 A1
(43) Pub. Date: Aug. 9, 2018

(54) A Torsion Spring Driven Injection Device

(71) Applicant: Novo Nordisk A/S, Bagsvaerd (DK)

(72) Inventors: Morten Revsgaard Frederiksen, Copenhagen K (DK); Lars Eilertsen, Fredensborg (DK); Ronan Carroll, Tisvildeleje (DK); Emil Gram Spork, Copenhagen N (DK)

(21) Appl. No.: 15/749,374

(22) PCT Filed: Jul. 29, 2016
(86) PCT No.: PCT/EP2016/068165

§ 371 Date: Jan. 31, 2018

(30) Foreign Application Priority Data
Jul. 31, 2015 (EP) 15179236.3

(51) Int. Cl. A61M 5/00; A61M 5/24; A61M 5/32; A61M 5/315; A61M 5/20

(52) U.S. Cl. CPC A61M 5/001; A61M 5/2422; A61M 5/3213; A61M 2005/3126; A61M 5/20; A61M 5/3155; A61M 5/31525

(57) ABSTRACT

The invention relates to a torsion spring driven injection device for apportioning individually set doses of a liquid drug. The injection device has a needle cannula (35) for multiple uses and a cleaning chamber (56) for cleaning the tip of the needle cannula between subsequent injections. The cleaning chamber is filled with preservative containing liquid drug directly from the cartridge (30) in a filling sequence which is executed when the user initiates first use of the injection device. The sequence comprises the step of securing the piston rod guide in an inrotatable position and thereafter moving the cartridge in a proximal direction. Such axial movement of the cartridge without a similar movement of the plunger automatically pumps liquid drug from the cartridge and into the cleaning chamber via the lumen of the needle cannula.

### THE TECHNICAL FIELD OF THE INVENTION

The invention relates to a medical injection device for injecting a liquid drug and especially to a pre-filled injection device for apportioning a multiple number of doses. The invention especially relates to such pre-filled injection device wherein the same needle cannula is used for a number of injections and wherein the tip of the needle cannula is cleaned between subsequent injections.

### DESCRIPTION OF RELATED ART

WO2015/062845 discloses an injection device in which the same needle cannula is used for multiple injections. The needle cannula is covered by a telescopically movable needle shield which also carries a cleaning chamber for cleaning the tip of the needle cannula between subsequent injections. The cleaning solvent inside the cleaning chamber is a quantum of the liquid drug contained in the cartridge of the injection device. The quantum of liquid drug is in one example (FIG. 8-9) transferred from the cartridge to the cleaning chamber by moving the cartridge axially a distance relatively to the plunger of the cartridge. This is done by maintaining the piston rod, the piston rod foot and thus the plunger in a fixed position while moving the cartridge in the proximal direction.

WO2014/060369 discloses an injection device in which the piston rod is able to move in either direction between injections in order to relief any pressure build up inside the cartridge. Such “pressure relief system” usually comprises a piston rod guide which is decoupled from the drive mechanism when an injection is not being performed.

However since the drive system is not locked, it is not possible to build up pressure inside the cartridge to transfer liquid drug to the cleaning chamber as the piston rod guide is decoupled and thus free to rotate.

### DESCRIPTION OF THE INVENTION

It is henceforth an object of the present invention to provide an injection device having a pressure relief system which can be disabled to transfer a quantum of liquid drug from the cartridge to the cleaning chamber.

It is further an object of a further invention to provide a mechanism which prevents the user form removing the cap from the injection device and thus perform an injection before the cleaning chamber has actually been correctly filled with liquid drug from the cartridge.

The invention is defined in claim 1. Accordingly, in a first aspect the present invention relates to a torsion spring driven injection device for automatically apportioning individually set doses of a liquid drug. The main components of such injection device are:
A housing storing a cartridge,
A needle cannula,
A movable needle shield,
A cleaning chamber,
A piston rod,
A piston rod guide,
A drive tube, and
A torsion spring.

The housing stores the cartridge containing the liquid drug to be ejected. The cartridge is mounted such that it can slide axially inside the housing,

The needle cannula, which is to be used for multiple injections, is connectable with the interior of the cartridge such that liquid drug can flow through the lumen of the needle cannula and the needle cannula further has a distal tip for penetrating the skin of a user during an injection.

The needle shield which in one preferred example is telescopically slidable or movable covers at least the distal tip of the needle cannula between injections. The movable needle shield further carries a cleaning chamber for cleaning the distal tip of the needle cannula between subsequent injections. The cleaning chamber can either be an integral part of the movable needle shield or coupled to the movable needle shield to form a movable needle shield assembly. The cleaning chamber is preferably filled with liquid drug from the cartridge.

Since the liquid drug contains a preservative, this preservative cleans the tip of the needle cannula between injections.

The piston rod has an outer surface with a thread and a not-circular cross-section. The not-circular cross section can be formed in many different ways, e.g. as a track or as a flattened longitudinal surface.

The rotatable piston rod guide either mates with the not-circular cross-section of the piston rod or has an inner thread mating the outer thread of the piston rod.

The drive tube is rotatable by the torsion spring at least during ejection of the dose and the torsion spring is operational provided between the housing and the drive tube to rotate the drive tube during ejection.

The multiple doses to ejected can either be predetermined by the manufacture of the injection device or the doses can be individually set by the user prior to performing the ejection.

The housing, or a housing element, is further operational provided with an internal opening either shaped as a thread mating the outer thread of the piston rod or as a key mating the not-circular cross-section of the piston rod. The piston rod is thus moved forward with or without rotation whenever the piston rod guide and the housing are rotated relatively.

The piston rod guide is axially movable at least between two positions;
a first position in which the piston rod guide operates independently of the drive tube e.g. by being decoupled from the drive tube, and
a second position in which the piston rod guide operates together with the drive tube e.g. by being coupled to the drive tube.

In the first position, the piston rod guide is operational decoupled from the drive tube such that the piston rod guide is free to rotate should the piston rod move axially. The pressure release mechanism is thus activated and axial movement of the plunger in the cartridge is conveyed to an axial movement of the piston rod and thus to a rotation of the piston rod guide.

In the second position, the pressure release mechanism is disabled by moving the piston rod guide axially into operational engagement with the drive tube which in this position is blocked. The piston rod guide is thus locked by the drive tube and unable to rotate.

Sliding the cartridge in the proximal direction when the piston rod guide is in the second position will thus create a pressure inside the cartridge since the piston rod, the piston rod foot and the plunger does not move axially. This pressure will thus force a quantum of the liquid drug contained inside the cartridge to flow through the lumen of the needle cannula and into the cleaning chamber.

Further, if the drive tube is released in the second position, the torsion spring will force the drive tube and the piston rod guide to rotate thus moving the piston rod forward toward the distal end of the cartridge.

The injection device is preferably a pre-filled injection device. Pre-filled injection devices are also often referred to as disposable injection device and means that the injection device is pre-filled with a specific amount of liquid drug. Once the liquid drug contained in the injection device has been used, the user discharges the entire injection device.

The pre-filled amount of liquid drug is usually contained in a cartridge which is permanently and non-removable embedded in the housing of the injection device such that both the housing and the cartridge are discarded together when the pre-filled injection device is disposed of.

In order to move the piston rod guide in the proximally direction from the first position to the second position, a release element is provided which release element preferably is operational by the movable needle shield, such that manipulation of the movable needle shield moves the release element proximally

The release element is preferably activated by a user during initiation of the injection device such that the piston rod guide is moved from the first position and into the second position in which the pressure release mechanism is disabled as the user makes the injection device ready for first use.

The piston rod guide is further moved in the distal direction from the second position to the first position by a resilient element.

Further, the release element is axially guided in the housing and connected to the movable needle shield by a thread. The release element is thus restricted to purely axial movement whereas the movable needle shield can be rotated preferably by the user. The movement here indicated shall be seen as being relatively to the housing.

As the movable needle shield is rotated it moves helically due to the threaded engagement with the release element which during initiation is temporarily locked to the housing.

In a further embodiment, the needle hub carrying the needle cannula is guided by the movable needle shield to move with the movable needle shield as the movable needle shield rotates. The needle hub is further guided helically such that the needle hub applies an axial pressure on the cartridge during its helical movement.

The pressure is preferably applied by having a tube-like structure on the needle hub in abutment with the distal end of the cartridge which is thereby pushed proximally as the needle hub is guided proximally.

The hub is preferably guided by an engagement with a helical track or the like such that the needle hub moves helically when pushed axially.

This axial pressure further moves the cartridge in the proximal direction and since the pressure relief mechanism is disabled thus preventing the piston rod guide from rotating, liquid drug will flow through the lumen of the needle cannula and into the cleaning chamber.

In a further aspect of the invention a mechanical user guiding mechanism is provided to guide the user correctly through a number of user steps required to transfer liquid drug from the cartridge and into the cleaning chamber. More specifically, the guiding mechanism comprises means preventing the user for removing the protective cap before a certain action has been done.

In one embodiment, the injection device comprises:

a housing having a first part and a removable cap at least partly covering the first part and removable in an axial direction of the injection device. Such cap is often referred to as a protective cap as it protects the distal end of the injection device.

The protective cap is internally provided with a first track extending substantially perpendicular to the axial direction, and the first part of the housing carries an outwardly pointing protrusion engaging the first track to prevent axial removal of the cap.

The outwardly pointing protrusion is however provided on a flexible arm which is able to bend, and a second part or element is provided adjacent the first part. Further, the second element is provided with a surface positioned radially to the flexible arm and which surface has at least to different levels;

a first level preventing radial movement of the flexible arm, and a second level allowing radial movement of the flexible arm. Further, means are provided to shift the two levels into position adjacent the flexible arm.

When the first level of the surface is placed radially to the flexible arm radial movement of the flexible arm is thus prevented and the outwardly poiting protrusion remains inside the first track thus preventing axial movement of the protective cap. However, when the second level is moved into position underneath the flexible arm, the arm is allowed to flex in the radial direction and so are the outwardly pointing protrusion such that the protective cap can be removed.

The second level is preferably a deepened surface area which lies lower than the first level such that the flexible arm is allowed to flex radially when the deepened area underlies the flexible arm which then is allowed to bend inwardly towards a centre line of the injection device. The protective cap is thus only able to slide off the injection device when the deepened area is located beneath the flexible arm.

In a pen-shaped injection device, the first track in the cap in which the outwardly pointing protrusion is axially blocked is preferably formed as a circumferentially track on an inside of the protective cap.

In a preferred example of this invention, the first part is hollow and the second element is rotatable contained at least partly within the first part. The first part being the housing and the second part e.g. being a rotatable movable needle shield. The deepened surface area of the second part can thus be brought into the radial proximity of the flexible arm carrying the outwardly protrusion by rotating the second part and the first hollow part relatively to each other.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel and connected to a needle hub to form a complete injection needle all though the needle cannula could also be connected directly to the housing structure without a hub. A needle cannula could however also be made from a polymeric material or a glass material.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

“Cleaning chamber” is in the present description broadly meant to be any kind of reservoir containing a cleaning solvent to clean at least the distal tip of the needle cannula between subsequent injections. Such cleaning chamber is preferably both distally and proximally sealed by a pierceable septum. However, the proximal septum could be replaced by any kind of sealing which would seal against the outer surface of the needle cannula. The distal septum and the proximal septum or seal of the cleaning chamber defines a confinement containing the cleaning solvent which cleaning solvent in a preferred embodiment is identical to the preservatives contained in the liquid drug used in the specific injection device. In a most preferred solution, the same preservative containing liquid drug is present in both the cleaning chamber and in the cartridge of the injection device thereby avoiding contamination of the preservative containing drug inside the cartridge.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

“Scale drum” is meant to be a cylinder shaped element carrying indicia indicating the size of the selected dose to the user of the injection pen. The cylinder shaped element making up the scale drum can be either solid or hollow. “Indicia” is meant to incorporate any kind of printing or otherwise provided symbols e.g. engraved or adhered symbols. These symbols are preferably, but not exclusively, Arabian numbers from “0” to “9”. In a traditional injection pen configuration the indicia is viewable through a window provided in the housing.

Using the term “Automatic” in conjunction with injection device means that, the injection device is able to perform the injection without the user of the injection device delivering the force needed to expel the drug during dosing. The force is typically delivered—automatically—by an electric motor or by a spring drive. The spring for the spring drive is usually strained by the user during dose setting, however, such springs are usually prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on, e.g. on the proximal end, of the injection device to release—fully or partially—the force accumulated in the spring when carrying out the injection.

The term “Permanently connected” as used in this description is intended to mean that the parts, which in this application is embodied as a cartridge and a needle assembly, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a perspective view of the injection device prior to use.

FIG. 2 show a perspective view with the protective cap moved proximally.

FIG. 3 show a perspective view when rotating the protective cap.

FIG. 4 show a perspective view with the protective cap removed.

FIG. 5 show an exploded view of the injection device.

FIG. 6A-B show a cross sectional view of the injection device prior to use.

FIG. 7A-C show the injection device with the cap axially moved.

FIG. 8 show a perspective view of the engagement between the needle hub and the housing.

FIG. 9 show a perspective view of the telescopically movable needle shield.

FIG. 10 show a perspective view of the release element.

FIG. 11 show a cross-sectional view of the injection device as rotation is initiated.

FIG. 12 show a cross-sectional view of the injection device with the cap rotated.

FIG. 13 show a cross-sectional view of the injection device in a ready-to-inject state.

FIG. 14 show a cross sectional view of the injection device of FIG. 13 with the protective cap removed.

FIG. 15A-B show a cross sectional view of the injection device during injection.

FIG. 16A-B show a cross sectional view of the injection device following an injection.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

In that context it may be convenient to define that the term “distal end” in the appended figures is meant to refer to the end of the injection device which usually carries the injection needle whereas the term “proximal end” is meant to refer to the opposite end pointing away from the injection needle and usually carrying the dose dial button.

Distal and proximal are meant to be along an axial orientation extending along the longitudinal axis “X” of the injection device and is further indicated in the figures.

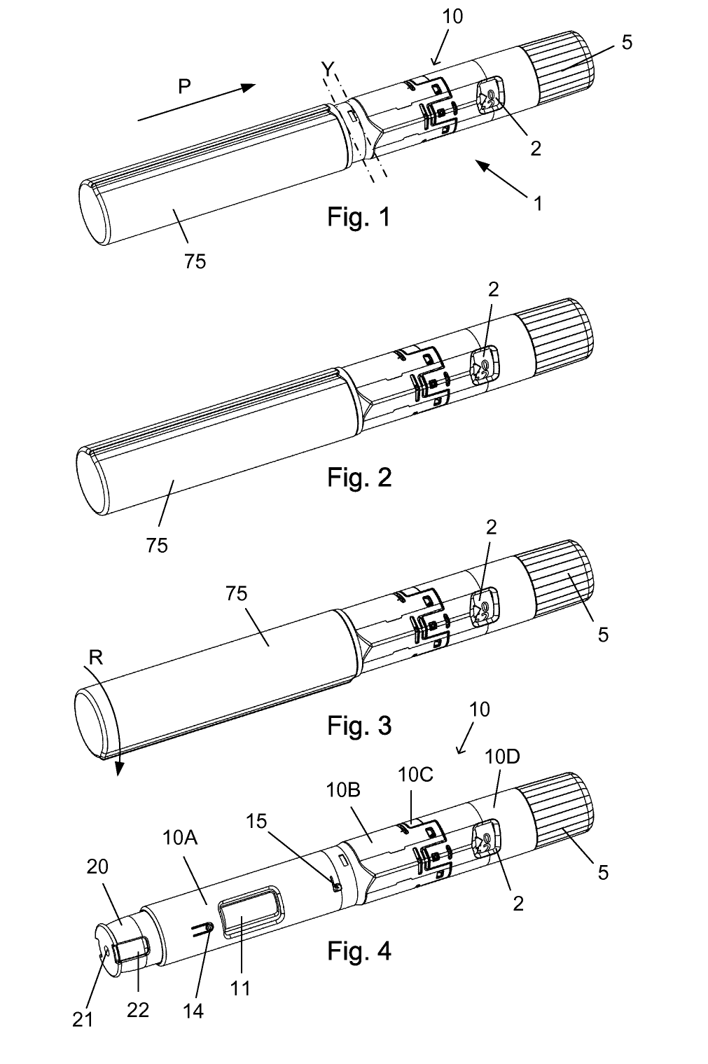

FIGS. 1 to 4 discloses the injection device according a first embodiment.

Proximally the injection device 1 is provided with a dose setting button 5 rotatable by a user to set a dose to be ejected. The dose setting button 5 is axially secured to the housing 10 such that the dose setting button 5 does not translate axially when rotated in either direction. The dose set by this rotation is visually shown on a scale drum 65 appearing in a dose window 2 provided in the housing 10.

The distal end of the injection device 1 is in FIGS. 1 to 3 covered by a removable protective cap 75 which can be manipulated by a user as will be explained.

FIG. 4 depicts the injection device 1 with the protective cap 75 removed. Distally a telescopically movable movable needle shield 20 covers the needle cannula 35. Further in FIG. 4, the protective cap 75 when mounted covers a longitudinal window 11 in the housing 10, through which longitudinal window 11, the user is able to visually inspect the drug contained in a cartridge 30 carried in the injection device 1.

The movable movable needle shield 20 is able to move telescopically and the distal end has an opening 21 through which the needle cannula 35 protrudes and is further provided with longitudinal tracks 22 which are engaged by gripping means provided internally in the protective cap 75.

The housing 10 is divided into a number of housing parts 10A, 10B, 10C, 10D which are click-fitted together to form one housing 10. In the embodiment disclosed in FIG. 1, four such housings parts 10A, 10B, 10C, 10D are shown. A distal housing part 10A, a proximal housing part 10D, a first intermediate housing part 10B and a second intermediate housing part 10C. Some of the housing parts 10A, 10B, 10C, 10D or all the parts could alternatively be moulded to form one or more unitary parts.

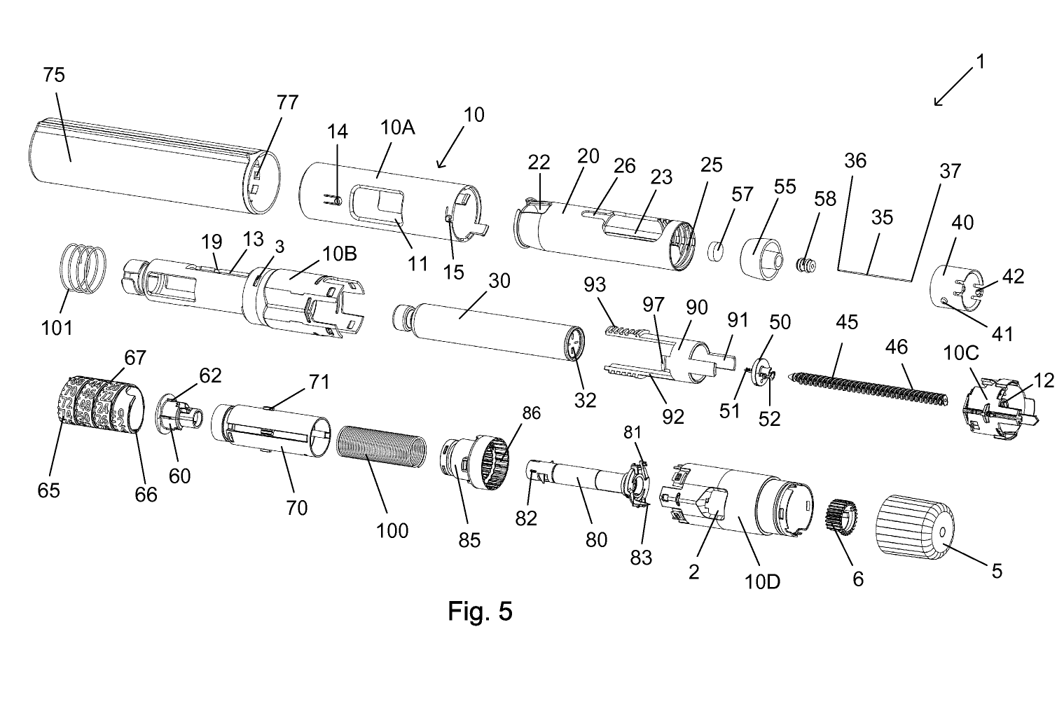

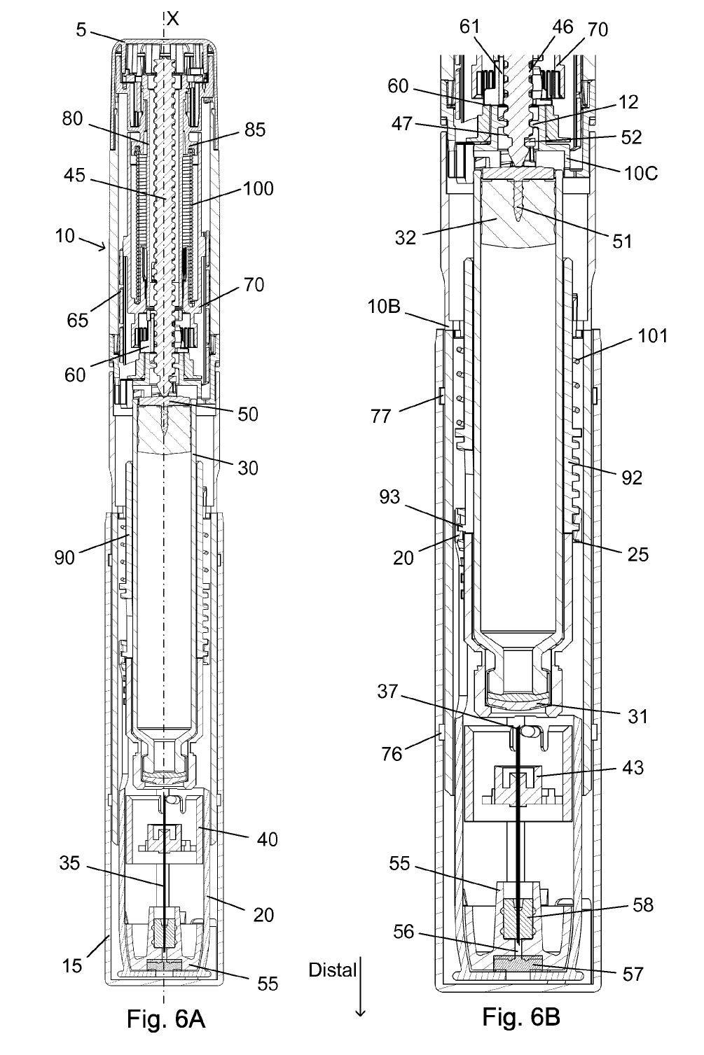

The second intermediate housing part 10C is internally provided with a thread 12 as best seen in FIG. 6B which depicts the injection device 1 as delivered to the user. An exploded view of the injection device 1 is disclosed in FIG. 5. The main components which are also identified on FIG. 6A are:
Dose setting button: 5
Housing: 10
Needle shield: 20
Cartridge: 30
Needle cannula: 35
Needle hub: 40
Piston rod: 45
Piston rod foot: 50
Cleaning capsule: 55
Piston rod guide: 60
Scale drum: 65
Drive tube: 70
Protective cap: 75
Ratchet element: 80
Spring base: 85
Release element: 90
Torsion spring: 100

The movable needle shield 20 is distally provided with a cleaning capsule 55 in which a cleaning chamber 56 is distally sealed by a distal septum 57 and proximally by a movable piston 58. The interior of the cleaning chamber 56 is, at least in a situation of use, filled with liquid drug from the cartridge 30 stored in the first intermediate housing part 10B as will be explained.

Further, a needle cannula 35 is provided. A distal end 36 of the needle cannula 35 is maintained inside the cleaning chamber 56 between injections and a proximal end 37 projects proximally from a hub 40 to which the needle cannula 35 is attached. The needle cannula 35 is hollow and a longitudinal lumen 38 connects the distal end 36 and the proximal end 37.

The first intermediate housing part 10B supports the cartridge 30 containing the liquid drug to be injected. The cartridge 30 is able to slide inside the first intermediate housing part 10B as will be explained. The distal end of the cartridge 30 is sealed by a pierceable septum 31 and the proximal end is sealed by a movable plunger 32. To expel liquid drug from the cartridge 30, a piston rod 45 carrying a piston rod foot 50 is moved distally inside the cartridge 30. The piston rod foot 50 abuts the plunger 32 inside the cartridge 30 thus forcing the plunger 32 forward such that the volume of the cartridge 30 containing the liquid drug is reduced and the liquid drug flows out through the lumen 38 of the needle cannula 35.

The piston rod foot 50 is provided with a spike 51 which connects the piston rod foot 50 to the plunger 32 such that the piston rod foot 50 and the plunger 32 move in unison. Proximally, the piston rod foot 50 is provided with connection means 52 connecting the piston rod foot 50 to the piston rod 45. The result being that the piston rod 45, the piston rod foot 50 and the plunger 32 move together in the axial direction

The piston rod 45 has an outer thread 46 which engages a corresponding thread 12 in the second intermediate housing part 10C such that whenever the piston rod 45 is rotated it screws forward or backwards. In order to rotate the piston rod 45 a piston rod guide 60 is provided which has an internal key 61 engaging a longitudinal track 47 provided in the piston rod 45.

To rotate the piston rod guide 60 a drive assembly is provided. This drive assembly comprises a drive tube 70 and a torsion spring 100 which is operable between the drive tube 70 and a spring base 85. The spring base 85 is firmly attached to the housing 10 in a non-movable manner, but could alternatively be moulded as an integral part of the housing 10. The torsion spring 100 is distally connected to the drive tube 70 such that the torsion spring 100 is strained when the drive tube 70 is rotated relatively to the housing 10 and the spring base 85.

The drive tube 70 is rotational connected to a scale drum 65 by having a protrusion 71 slidable engaged in a longitudinal track 66 provided inside the scale drum 65. The scale drum 65 further has a helical outer track 67 which travels in a corresponding thread inside the housing 10 such that scale drum 65 travels helically when rotated by the drive tube 70.

In order to strain the torsion spring 100, a ratchet element 80 connects the drive tube 70 with the dose setting button 5. Rotation of the dose setting button 5 is thus via a gearing wheel 6 transferred to a rotation of the drive tube 70 and thus a straining of the torsion spring 100. The connection between the dose setting button 5 and the ratchet element 80 is provided such that the dose setting button 5 and the drive tube 70 can be rotated in both rotational directions.

The dose dial button 5 is connected to the ratchet element 80 via a ratchet mechanism as described in WO 2013/178372 which allows the dose setting button 5 to be rotated in both directions while the ratchet mechanism holds the torque of the torsion spring 100 until the set torque is released. The ratchet mechanism essentially comprises ratchet arms 81 operating in a toothed ring 86 of the spring base 85, such that the ratchet element 80 is held in its position when the torsion spring 100 is strained. For that purpose, the ratchet element 80 is distally provided with a plurality of teeth 82 engaging a toothing provided internally in the drive tube 70. Further, the dose setting button 5 is internally provided with teeth which are able to move the ratchet arms 81 radially whenever the dose setting button 5 is rotated in the dose reducing direction. This allows the ratchet element 80 to move rotational backward in relation to the toothed ring 86 such that the set dose can be incrementally reduced.

FIG. 6A discloses the situation in which the injection device 1 has not been taken into use. FIG. 6B is an enlarged picture of a part of the injection device 1 shown in FIG. 6A. In this situation, the piston rod guide 60 is decoupled from the drive tube 70 such that the piston rod guide 60 is free to rotate. The piston rod guide 60 rotates freely on a proximal extension of the housing part 10C which proximal extension internally carries the thread 12 for the piston rod 45.

Should the liquid drug inside the cartridge 30 e.g. be exposed to temperature changes, the volume will expand or retract and the plunger 32 will henceforth move axially. This axial movement of the plunger 32 is conveyed to the piston rod foot 50 which moves in unison with the plunger 32. Due to the connection, via the connecting means 57, between the piston rod foot 50 and the piston rod 45, the piston rod 45 will also move axially. Since the piston rod 45 is threadely connected to the thread 12 of the housing part 10C, the piston rod 45 will rotate as it is moved axially. This rotation of the piston rod 45 makes the piston rod guide 60 rotate as the key 61 provided inside the piston rod guide 60 engages the longitudinal track 47 of the piston rod 45.

As the piston rod guide 60 in this situation is not rotational locked it rotates freely as the piston rod 45 move in either direction. Since the piston rod 45 and the piston rod foot 50 are secured to the plunger 32, the piston rod 45 will follow any movement of the plunger 32. The pressure inside the cartridge 30 will therefore automatically adjust to the temperature simply by rotation of the piston rod guide 60. This principle is referred to as pressure relief.

To release the torsion spring 100 to rotate the piston rod guide 60 and thereby the piston rod 45, a release element 90 is provided. As disclosed in FIG. 10, this release element 90 has a plurality of release arms 91 extending in a proximal direction and further a plurality of thread-arms 92 projecting in the distal direction. These thread-arms 92 are distally provided with a guiding thread 93. At least one of the thread-arms 92 is divided such that a trigger arm 94 also pointing in the distal direction is provided. This trigger arm 94 carries a trigger knob 95, the function of which will be explained latter. Further, the trigger-arm 94 is distally provided with a thread segment 96 that follows the pitch of the guiding thread 93. The release element 90 is on a sidewall also provided with an outwardly pointing protrusion 97, the function of which will be explained latter. The proximal ridge 98 of the guiding thread 93 further forms a basis for a compression spring 101.

The compression spring 101 lies between the ridge 98 of the guiding thread 93 on the release element 90 and the first intermediate housing part 10B urging the release element 90 in the distal direction. The compression spring 101 is slightly pre-tensed such that the guiding element 90 is constantly urged in the distal direction.

The release element 90 operates inside the first intermediate housing part 10B and the guiding thread 93 is guided in a longitudinal track 13 provided in first intermediate housing part 10B such that release element 90 only travels axially. The guiding thread 93 protrudes through the opening 13 and is engaged by the internal track 25 of the movable needle shield 20 which slides on an outside surface of the first intermediate housing part 10B

When the injection device 1 is delivered to a user, the cap 75 is pre-mounted, the cleaning chamber 56 is empty and the proximal end 37 of the needle cannula 35 is not inserted into the cartridge 30. This is the situation depicted in FIG. 1 and in FIG. 6. The user initially has to prepare the injection device accordingly before an injection can be performed. During these preparations, the pierceable septum 31 of the cartridge 30 must be penetrated by the proximal end 37 of the needle cannula 45 and the cleaning chamber 56 must be filled with liquid drug from the cartridge 30.

When initiating the injection device, the user first moves the protective cap 75 a distance “Y” in the proximal direction in a purely axial movement as depicted in FIG. 1. This axial movement also move the movable needle shield 20 in the proximal direction since the distal end of the movable needle shield 20 abut the interior of the protective cap 75.

The movable needle shield 20 is on the interior surface provided with a longitudinal track 23 which track 23 is provided with a radial parking track 24 (see FIG. 9). In the initial position of the needle shield 30, a protrusion 41 provided on an outer wall of the hub 40 is parked in this radial parking track 34 such that the hub 40 follows axial movement of the needle shield 30.

When delivered to the user as depicted in FIG. 6A-B, the internal thread 25 of the movable needle shield 20 is engaged with the distal end of the guide thread 93 of the thread arms 92 of the release element 90 such that the movable needle shield 20 and the guiding element 90 move axially together guided by the longitudinal opening 13.

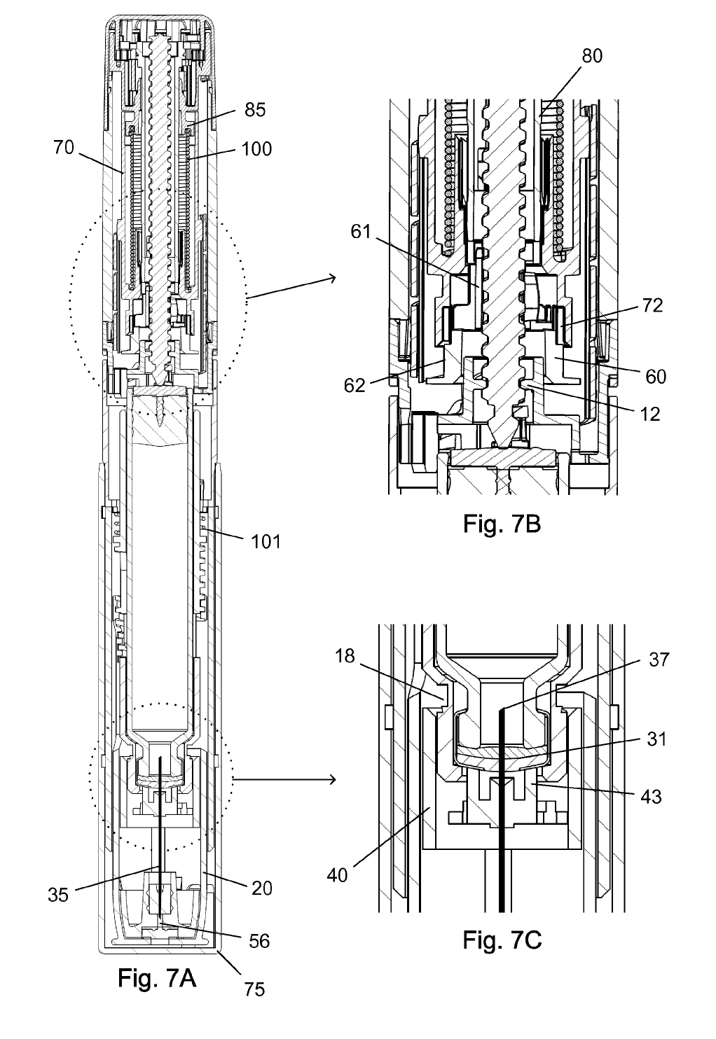

When the user initates use of the injection, the user pushes the protective cap 75 in the proximal direction. This purely axial movement is conveyed to the movable needle shield 20 which thus also moves axially together with guiding element 90. This axial movement further tightens the compression 101 as disclosed in FIG. 7A.

As the guiding element 90 is moved axially, the trigger-knob 95 slides axially along the side of the longitudinal opening 13 and is caught by the indentation 19 provided in the side wall of the longitudinal opening 13 thus temporary securing the guiding element 90 in this position.

The protective cap 75 is internally provided with a first track 76 and a second track 77. When the protective cap 75 is pushed proximally, the first track 76 engages a first protrusion 14 provided externally on the first housing part 10A and the second track 77 engages a second protrusion 15 also provided on the first housing part 10A. Both engagements provide tactile information to the user that the protective cap 75 has reached its correct proximal position.

Both the first protrusion 14 and the second protrusion 15 are provided on individual flexible arms which can be hindered in radial movement by placing a solid element under the flexible arms. The movable needle shield 20 is e.g. provided with a longitudinal recess 26 such that the flexible arm carrying the first protrusion 14 can only flex radially when the flexible arm is located above this recess 26. The protective cap 75 can thus only be axially moved on the first housing part 10A when the movable needle shield 20 is in a specific rotational position.

Further in the position disclosed in FIG. 7A-C, the protrusion 97 is positioned directly underneath the opening 3 in the first housing part 10B. At the same time, the flexible arm carrying the protrusion 15 is located radially above the opening 3. The result being that the arm carrying the protrusion 15 can only flex very limited. At the same time the track 77 is formed with a plurality of steep flanges in one rotational direction such that the protective cap 75 can only rotate in one rotational direction.

In the proximal position of the protective cap 75 as disclosed in FIG. 7A-C, the hub 40 has been moved together with the movable needle shield 20 and the proximal end 37 of the needle cannula 35 is now inserted into the cartridge 30 (see FIG. 7C).

The FIGS. 7B and 7C are partial enlargement of details in FIG. 7A. As the movable needle shield 20 moves proximally it brings along the release element 90 due to the engagement between the internal thread 25 of the movable needle shield 20 and the external thread 93 on the thread arms 92. In the position shown in FIG. 7A-C, in which the protective cap 75 is correctly positioned in its proximal position, the knob 95 is engaged in the indentation 19 and the compression spring 101 is fully compressed.

The axial movement of the release element 90 is via the release arms 91 transferred to an axial movement of the piston rod guide 60 such that external teeth 62 provided externally on the piston rod guide 60 engages an internal toothing 72 inside the drive tube 70 (see FIG. 7B). The piston rod guide 60 is hereafter rotational locked to drive tube 70 with the result that the piston rod 45 cannot rotate any longer. The pressure relief system is in this manner disabled and the piston rod 45 is unable to flow freely.

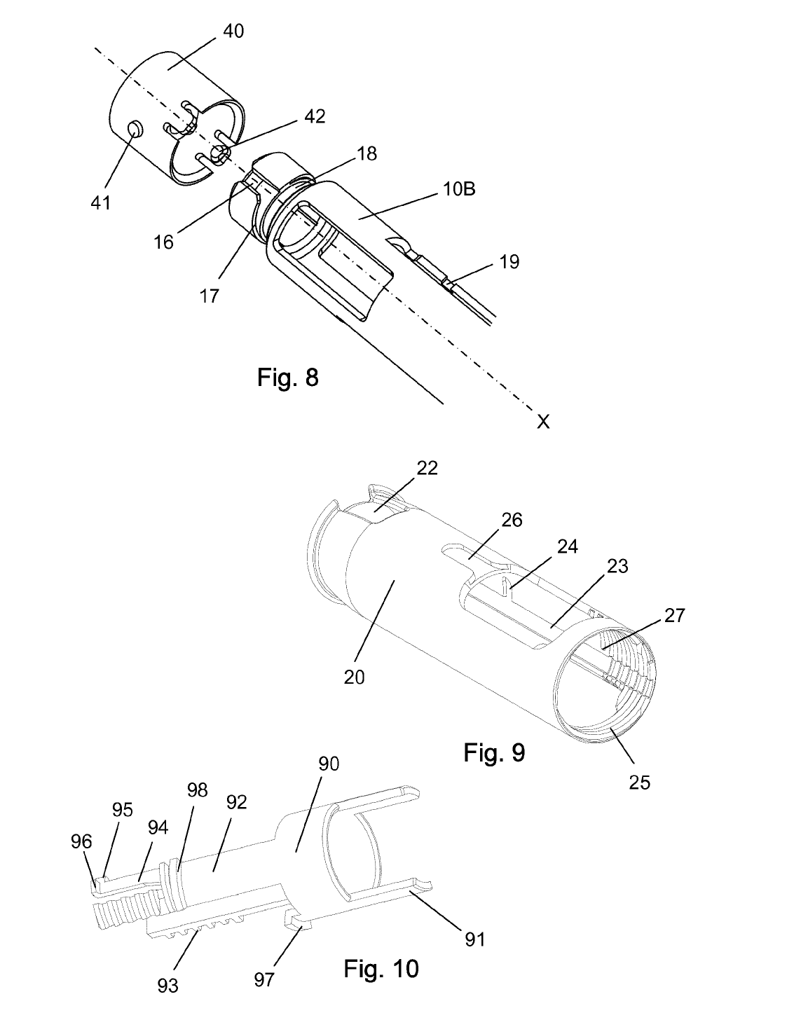

When moving the movable needle shield 20 and the release element 90 proximally, the needle hub 40 which is locked in the radial parking track 24 also moves proximally. As disclosed in FIG. 8, the hub 40 is internally provided with inner protrusions 42 which during the proximal movement of the needle hub 40 slides axially in axial tracks 16 provided in the first intermediate housing part 10B. As long as the inner protrusions 43 is positioned in the axial tracks 16, the hub 40 is unable to rotate.

Following the axial movement of the movable needle shield 20 which is finalized when the first track 76 engages with the first protrusion 14 as disclosed in FIG. 2, the user starts rotating the protective cap 75 as indicated by the arrow “R” in FIG. 3. The connection between the second protrusion 15 and the second track 77 is as previously explained formed such that the protective cap 75 can only rotate in one rotational direction. The rotational direction being one that screws the movable needle shield 20 in the proximal direction in the guiding thread 93.

As the movable needle shield 20 is rotated and translated in the proximal direction the side surface of the longitudinal track 23 abuts the protrusion 41 of the needle hub 40 which is thereby also rotated. During the rotation of the hub 40 the internal protrusions 42 abut a sloping surface 17 in the axial track 16 which forces the hub 40 to move in the proximal direction.

The hub 40 is internally provided with an internal sleeve 43 which are best seen in FIG. 7C. The sleeve is hollow and surrounds the needle cannula 35. The hollow sleeve 43 could alternatively be formed from a number of axially extending arms. The proximal end of the hollow sleeve 43 abut the distal end of the cartridge 30 and as the hub 40 travels in the proximal direction the sleeve 43 pushes the cartridge 30 also in the proximal direction.

For that purpose, the cartridge 30 is supported in the first intermediate housing part 10B in a way allowing axial movement of the cartridge 30.

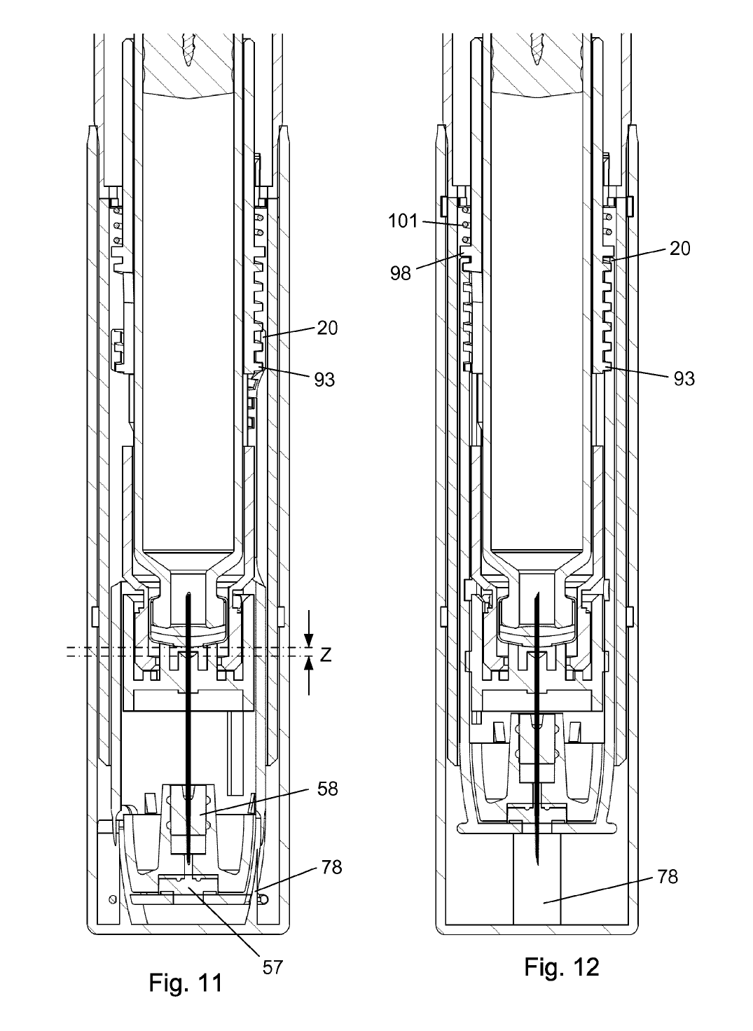

As the movable needle shield 20 is rotated relatively to the guiding element 90, the internal protrusion 42 slides along the slope 17 while the cartridge 30 moves a distance “Z” in the proximal direction as disclosed in FIG. 11. Once the cartridge has moved the distance “Z” in the proximal direction, the internal protrusions 42 enters into the circular track 18 which allows full rotation of the hub 40.

Since the piston rod guide 60 is rotational locked to the drive tube 70 in this situation, the piston rod foot 50 and thus the plunger 32 is prevented from moving in the proximal direction. However, as the cartridge 30 is moved proximally a pressure will build up internally in the cartridge 30 which pressure will force liquid drug to be pressed through the lumen 38 of the needle cannula 35.

Since the distal tip 36 of the needle cannula 35 is maintained inside the cleaning chamber 56 this chamber 56 will be filled with liquid drug and the piston 58 will move in the proximal direction thus allowing the cleaning chamber 56 to be filled.

As the cartridge 30 is moved the distance “Z” in the proximal direction, and the cleaning chamber 56 is filled an overpressure can be build inside the cartridge 30. In order to equalize such overpressure, the user keeps rotating the protective cap 75 and thus the movable needle shield 20 due to the engagement between the track 22 and a corresponding axial ridge 78 provided inside the protective cap 75. Due to this further rotation of the protective cap 75 and the movable needle shield 20, the movable needle shield 20 screws proximally in the thread 93 of the guiding element 90.

When the thread 25 internally in the needle shield reaches the end of the thread 93 of the guiding element 90, the distal end 36 of the needle cannula 35 has penetrated through the septum 57 of the cleaning chamber 56 thus allowing an overpressure inside the cartridge 30 to escape. This is depicted in FIG. 12 where it is seen that the movable needle shield 20 is moved to its proximal end position as the thread 93 has reached the end of the thread 25.

As best seen in FIG. 9, the internal thread 25 of the movable needle shield 20 has an abrupt end 27. Once the thread segment 96 on the trigger arm 94 abuts this abrupt end 27, the trigger arm 94 will be forced to bend in the peripheral plane and thus be moved out of its engagement with the indentation 19 in the longitudinal opening 13.

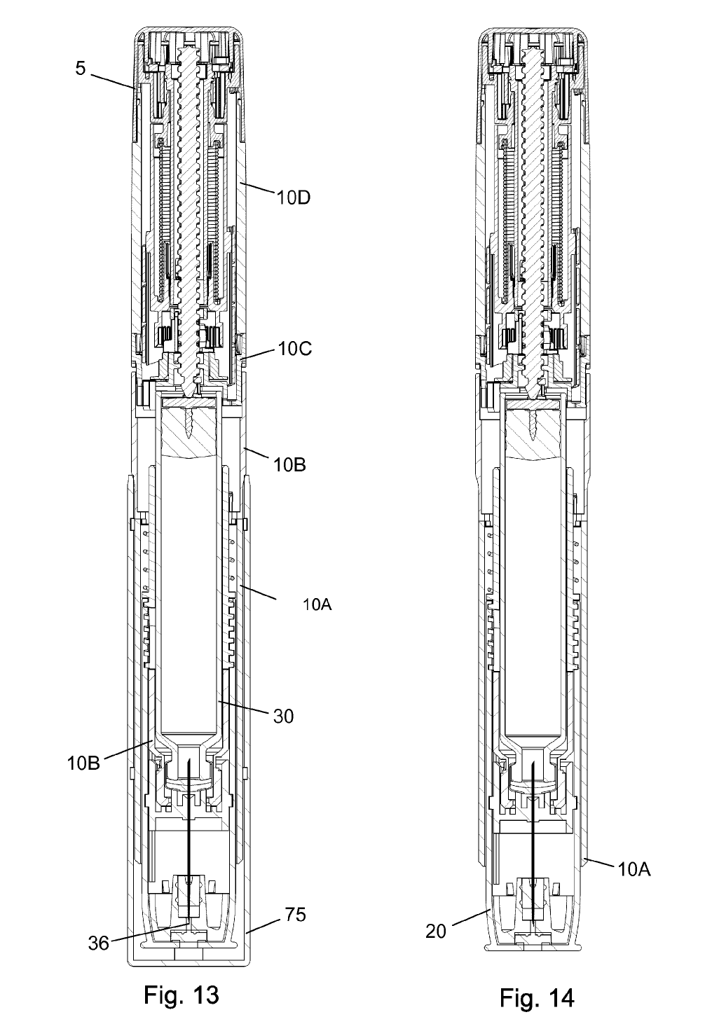

Once the trigger-knob 95 is moved radially free of the indentation 19, the compression spring 101 will force the movable needle shield 20 and the release element 90 to move proximally into the position shown in FIGS. 13 and 14.

As the movable needle shield 20 and the release element 90 move distally, a spring arm 83 provided proximally on the ratchet element 80 will move both the ratchet element 80 and the piston rod guide 60 distally as also shown in FIG. 13 and FIG. 14 wherein FIG. 14 is identical to FIG. 13 however with the protective cap 75 removed. In this position, the injection device 1 is ready to perform an injection. The cleaning chamber 56 is filled and the piston rod guide 60 is in the pressure relief position i.e. the piston rod 45 floats freely inside the injection device to compensate for temperature changes.

Also in the position disclosed in FIG. 13, the first flexible arm carrying the first protrusion 14 is located radially above the 26 such that the flexible arm can flex and the protective cap can be axially removed.

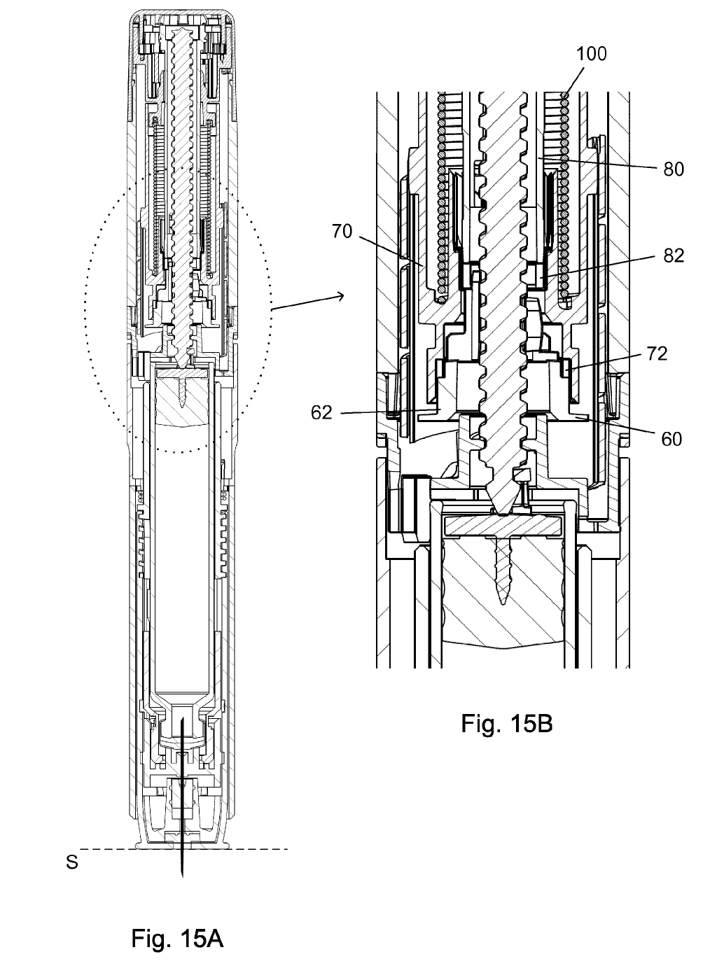

With the injection device 1 in the position disclosed in FIG. 13, the user rotates the dose setting button 5 to set a dose to be injected. During this rotation, the torsion spring 100 is strained. The user then removes the protective cap 75 as shown in FIG. 14 and presses the distal end of the movable needle shield 20 against the skin as indicted by the dotted line “S” in FIG. 15A.

As the movable needle shield 20 is locked to the guiding element 90 these two elements 20, 90 translates axially in unison which tightened the compression spring 101.

The axial movement of the guiding element 90 is translated to the piston rod guide 60 which also move axially as depicted in FIG. 15B. The teeth external 62 on the piston rod guide 60 thus engages the internal toothing 72 in the drive tube 70 which renders the piston rod guide 60 inrotatable in relation to the drive tube 70.

At the same time the proximal end of the piston rod guide 60 pushes the ratchet element 80 in the proximal direction such that the teeth 82 provided distally on the ratchet tube slides out of its engagement with the drive tube 70. As the drive tube 70 is no longer secured against rotation, the torsion spring 100 forces the drive tube 70 to rotate. Since the piston rod guide 60 in this position is inrotatable coupled to the drive tube 70, the piston rod guide 60 rotates with the ratchet element 80 and this rotation is translated to a rotation of the piston rod 45 which is thus screwed forward in its threaded connection 12/46 with the housing 10.

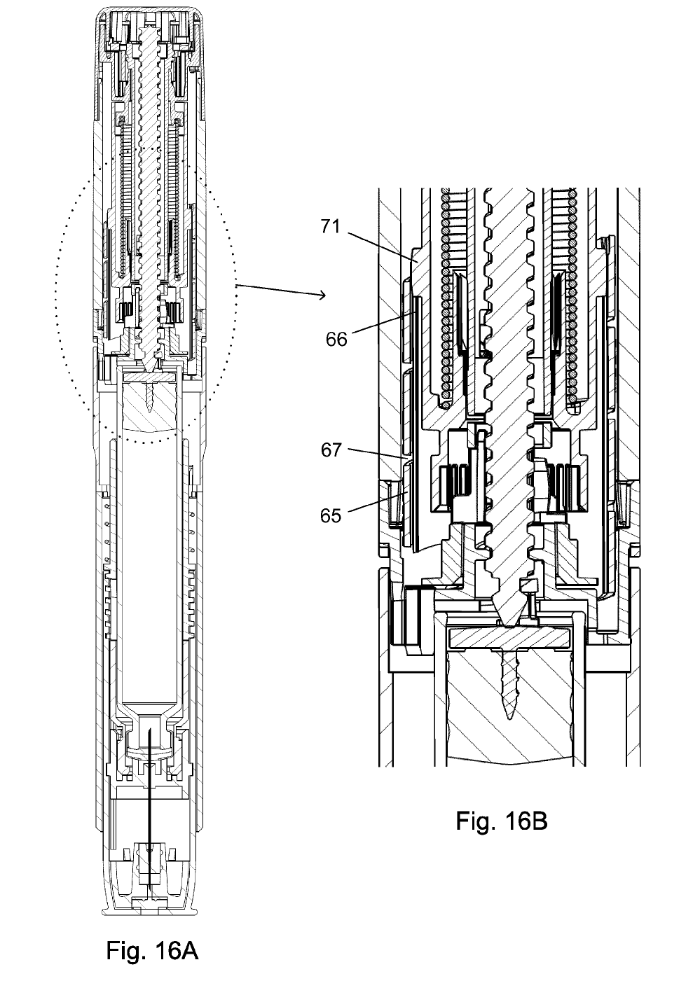

Once the liquid drug in the set dose has been delivered to the user, the needle cannula 35 is removed from the skin and the compression spring 101 urges the movable needle shield 20 and the guiding element 90 in the distal direction as shown in FIG. 16A-B which is actually the same situation as disclosed in FIG. 14.

In this position the spring arm 83 provided proximally on the ratchet element 80 moves the ratchet element 80 distally such that the teeth 82 re-enters the engagement with the drive tube 70 thus securing the torsion spring 100. This movement also moves the piston rod guide 60 into its pressure relief position wherein the piston rod guide 60 is free to rotate under influence of the axial movement of the plunger 32.

As the movable needle shield 20 slides back to its initial position, the distal tip 36 of the needle cannula 35 re-enters the cleaning chamber 56 such that the distal tip 36 is contained submerged in the liquid drug contained in the cleaning chamber 56 until next injection.

Since the liquid drug both in the cartridge 30 and especially in the cleaning chamber 56 contains a preservative, the distal tip 36 of the needle cannula 35 is biological cleaned before the next injection.

Some preferred embodiments have been shown in the foregoing, but it should be stressed that the invention is not limited to these, but may be embodied in other ways within the subject matter defined in the following claims.

1. A torsion spring driven injection device for apportioning multiple doses of a liquid drug, comprising:
a housing storing a cartridge containing the liquid drug,
a needle cannula connectable with the interior of the cartridge and having a distal tip needle shielded by a movable needle shield carrying a cleaning chamber for cleaning the distal tip of the needle cannula between subsequent injections,
a piston rod having an outer surface with a thread and a not-circular cross-section,
a rotatable piston rod guide mating the not circular cross-section of the piston rod or having an inner thread mating the outer thread of the piston rod, and
operationally provided in the housing; an internal thread mating the outer thread of the piston rod or a cross-section mating the not circular cross-section of the piston rod such that the piston rod is moved forward in relation to the housing when the piston rod guide are rotated relatively to the housing,
a drive tube which is rotatable by a torsion spring operational positioned between the housing and the drive tube,
wherein the piston rod guide is axially movable between;
a first position in which the piston rod guide operates independently of the drive tube, and
a second position in which the piston rod guide operates together with to the drive tube.

2. A torsion spring driven injection device according to claim 1, wherein the injection device is a pre-filled injection device wherein the cartridge is permanently embedded in the housing.

3. A torsion spring driven injection device according to claim 2, wherein the piston rod guide is movable in the proximally direction from the first position to the second position by engagement with a release element.

4. A torsion spring driven injection device according to claim 2, wherein the piston rod guide is movable in the distal direction from the second position to the first position by a resilient element.

5. A torsion spring driven injection device according to claim 2, wherein the release element is axially guided in the housing and threadedly connected to the telescopically movable needle shield.

6. A torsion spring driven injection device according to claim 5, wherein the telescopically movable needle shield is rotatable relatively to the housing and to the release element.

7. A torsion spring driven injection device according to claim 6, wherein the needle hub is guided helically during rotation of the movable needle shield.

8. A torsion spring driven injection device according to claim 7, wherein the needle hub when moved helically applies an axial pressure on the cartridge.

9. A torsion spring driven injection device according to claim 8, wherein the axial pressure moves the cartridge in the proximal direction.

10. A torsion spring driven injection device according to claim 3, wherein the piston rod guide is prevented from rotation when the release element is moved proximally.

11. An injection device comprising:
a housing comprising a first part, and
a removable protective cap partly covering the first part and removable in an axial direction (X), wherein
the protective cap internally is provided with a track extending substantially perpendicular to the axial direction (X), and the first part carries an outwardly pointing protrusion engaging the track to thereby prevent axial removal of the cap, which outwardly pointing protrusion is provided on a flexible arm, and wherein a second element is provided radially adjacent the first part and which second element is provided with a surface positioned radially to the flexible arm and which surface has at least to different levels;
a first level preventing radial movement of the flexible arm and the outwardly pointing protrusion, and
a second level allowing radial movement of the flexible arm and the outwardly pointing protrusion and wherein means are provided to shift the two levels into position radially adjacent to the flexible arm carrying the outwardly pointing protrusion.

12. An injection device according to claim 11, wherein the injection device is pen-shaped and the track is circumferentially provided inside the protective cap.

13. An injection device according to claim 12, wherein the first part is hollow and the second element is rotatable contained inside the first part.

14. An injection device according to claim 13, wherein the second element has an area radially distanced from the flexible arm such that the flexible arm is radially movable into this area when the area is brought into the radial proximity of the flexible arm carrying the protrusion by rotating the second element and the first hollow part relatively to each other.
